(19) United States
(12) Patent Application Publication
(10) Pub. No.: US 2005/0053517 A1
(43) Pub. Date: Mar. 10, 2005

(54) Sanitizing food products with recirculating dewatering stream

(76) Inventors: Donald S. Finan, Palos Park, IL (US); Richard Gaber, Clarendon Hills, IL (US)

Correspondence Address: PRAXAIR, INC.;LAW DEPARTMENT - M1 557, 39 OLD RIDGEBURY ROAD, DANBURY, CT, 06810-5113, US

(21) Appl. No.: 10/654,935

(22) Filed: Sep. 5, 2003

(51) Int. Cl. A61L 2/18

(52) U.S. Cl. 422/31; 422/300

(57) ABSTRACT

Product such as food is sanitized by spraying a sanitizing solution oonto it, drawing excess liquid off of the product by directing at the product a gaseous stream, dewatering the drawn-off stream, and recycling the dewatered stream for to be directed at additional product.

### FIELD OF THE INVENTION

The present invention relates to sanitizing products, especially food products.

### BACKGROUND OF THE INVENTION

A popular method for sanitizing food products before they are packaged and sold for consumption is to spray an ozone-water solution or mixture onto the surface of the food product. The ozone is a safe and effective agent for sanitizing the food surfaces. Using any water-borne sanitizing agent, such as an ozone solution, introduces liquid onto and into the product. It is highly desirable to remove this liquid, as otherwise it can contribute to the food product becoming soggy or wilted with the passage of time.

One technique frequently employed to remove water from the food product is to direct a high velocity air stream toward and past the food product, to blow the excess water off of the food pieces. Whether the air stream is applied as a jet of air blown from above, or is drawn past the food product by application of relatively high vacuum typically from below the food product, such techniques have several drawbacks. One drawback is that they are very energy intensive. Another drawback is that this air may contain ozone which must pass through a destruct unit before exhausting to the atmosphere. Another drawback is that the air that is employed needs to be as aseptic as possible, and particle-free, so that the air stream does not re-introduce onto the surfaces of the food product harmful agents which the ozone treatment was intended to inactivate. However, treating the air so that it is satisfactorily aseptic and particle-free is also expensive and requires maintenance of relatively complex devices in close proximity to the food sanitizing machinery. In addition, it is preferable that air used for this purpose is relatively cool, and the refrigeration equipment that is necessary to keep the air at cooled temperatures despite what may be higher ambient temperatures is also expensive and difficult to maintain.

Thus, there remains a need for sanitizing products such as food products, employing water-borne sanitizing agents, wherein the remaining water can be removed in a manner which leaves the food product sanitized but which is less expensive and less cumbersome to operate.

### BRIEF SUMMARY OF THE INVENTION

One aspect of the invention is a method for sanitizing an article comprising
(a) providing a housing having an entrance through which an article to be sanitized can enter the housing, an exit from which the article can exit the housing, and means for transporting the article within said housing from said entrance to said exit,
(b) applying an aqueous sanitizing solution comprising a sanitizing agent to the article within said housing,
(c) directing a gaseous stream into contact with a surface of the article within said housing and drawing said gaseous stream past a surface of said article in a manner which draws water that was applied in step (b) off of the article and into the gaseous stream drawn past said article, to form an off-stream comprising said gaseous stream and said water drawn off of said article,
(d) removing liquid water from said off-stream, thereby producing a dewatered gaseous stream,
(e) recycling said dewatered gaseous stream to step (c) to comprise the gaseous stream directed toward a surface of the article in step (c),
(f) cooling one or both of said dewatered gaseous stream and said water drawn off of said article as necessary to prevent the temperature of said gaseous stream from exceeding a predetermined maximum temperature, and
(g) withdrawing a gaseous side stream from said dewatered gaseous stream within said housing as necessary to enable the atmospheric pressure within said housing to be less than the atmospheric pressure outside said housing, and discharging said side stream outside said housing.

Another aspect of the present invention is apparatus useful for sanitizing an article comprising
(a) a housing having an entrance through which an article to be sanitized can enter the housing, an exit from which the article can exit the housing, and means for transporting the article within said housing from said entrance to said exit,
(b) means for applying an aqueous sanitizing solution comprising a sanitizing agent to the article within said housing,
(c) means within said housing for directing a gaseous stream into contact with a surface of the article to which said aqueous sanitizing solution has been applied,
(d) means for drawing said gaseous stream past a surface of said article in a manner which draws water off of the article and into the gaseous stream drawn past said article, to form an off-stream comprising said gaseous stream and said water drawn off of said article,
(e) means for removing liquid water from said off-stream, thereby producing a dewatered gaseous stream,
(f) means for recycling said dewatered gaseous stream to step (c) to comprise the gaseous stream directed toward a surface of the article in step (c),
(g) (i) means for cooling said dewatered gaseous stream, or (ii) means for cooling and said water drawn off of said article, or (iii) both means (i) and means (ii), wherein said means (i), (ii) or (iii) is capable of cooling sufficiently to prevent the temperature of said gaseous stream from exceeding a predetermined maximum temperature, and
(h) means for withdrawing a gaseous side stream from said dewatered gaseous stream within said housing as necessary to enable the atmospheric pressure within said housing to be less than the atmospheric pressure outside said housing, and for discharging said side stream outside said housing.

As used herein, the term “food product” is meant to include any edible vegetable or animal-source product, cooked or uncooked, whether or not intact or already subdivided into portions or pieces, and includes products which are added to preparations made from food products but which are themselves not necessarily consumed, such as spices, seasonings, and herbs. Examples include, without limitation, whole animal carcasses, portions of animal carcasses, individual mouth-sized pieces cut from animal carcasses, whole vegetables and fruits, pieces of vegetables, and pieces of fruits.

As used herein the term “sanitizing” means reducing or eliminating one or more of bacteria, microbes, molds, yeast and food spoilage enzymes from a product.

### BRIEF DESCRIPTION OF THE DRAWING

FIG. 1 is a perspective view of a housing within which the apparatus of FIG. 2 can be situated.

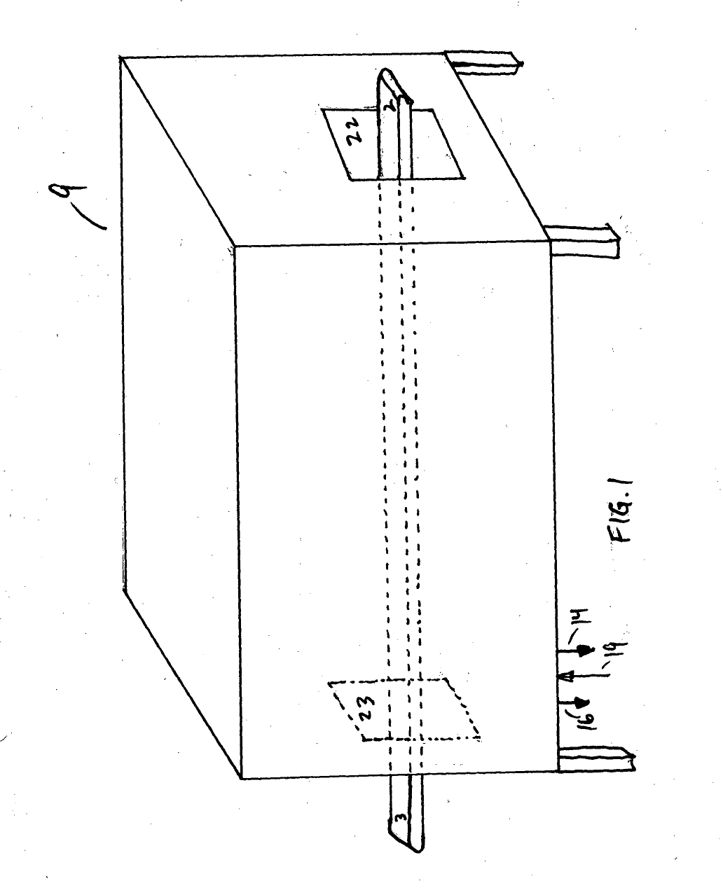

FIG. 2 is a schematic view of apparatus useful in the method of the present invention.

### DETAILED DESCRIPTION OF THE INVENTION

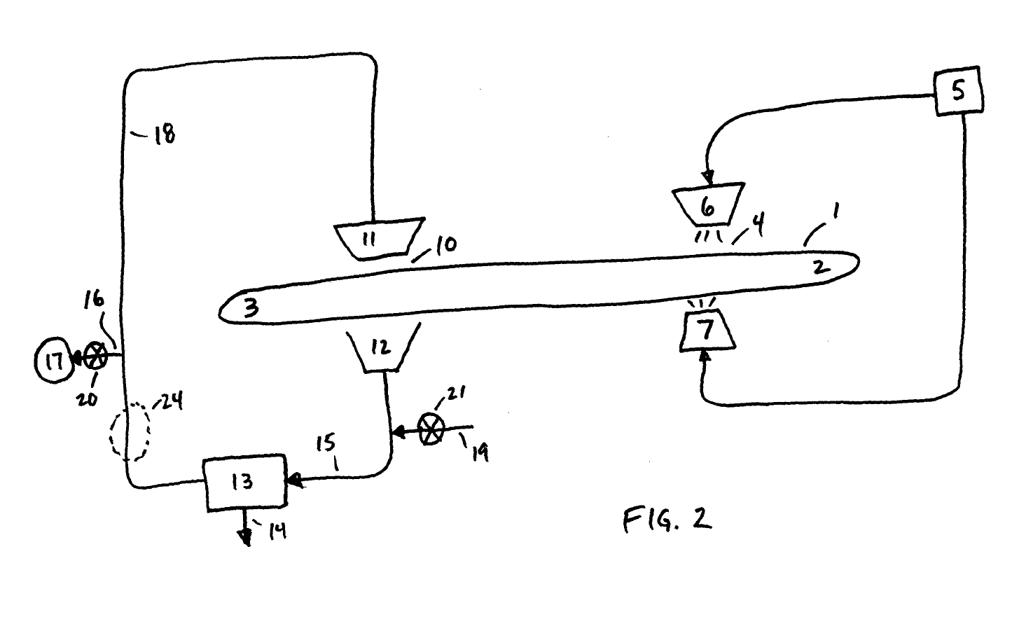

The invention is conveniently practiced using apparatus such as depicted in the Figures. Housing 9 of any conventional construction encloses conveyor 1 and the apparatus that is depicted in FIG. 2. Conveyor 1 of conventional construction moves the article(s) to be sanitized in a direction from end 2, where articles enter the housing through entrance 22, to end 3, where articles exit the housing through exit 23.

In the first stage of this invention, a sanitizing liquid is applied at station 4 to a surface (and preferably to all accessible surfaces) of the food product. The preferred technique is to spray sanitizing liquid onto all external surfaces of the food product. The sanitizing liquid can be prepared in, and pumped from, station 5.

The sanitizing liquid contains one or more compounds effective to inactivate food microbes present on the food product. “Agent” herein includes individual such compounds and also includes mixtures thereof. An agent that would be useful in this invention must leave no toxic residue on the food product, must be approved by governmental regulations for use on food products, and must be effective to inactivate microbes. Many such agents are known in the food treatment field. Examples include compounds of chlorine or bromine, examples of which include HOCl and ClO₂, peroxides, peracetates and peracetic acid, edible organic acids, of which the preferred examples are acetic, ascorbic, malic, lactic and citric acids, and edible inorganic acids and salts such as phosphoric acid and its sodium salts such as trisodium phosphate.

The concentration of the agent should be that which is effective to inactivate microbes. The effective concentration will vary with various agents, and with the length of time that the agent is in contact with the food product, but the effective concentration can readily be determined from published sources or by routine testing. Amounts can be from 50 or even 100 ppm up to several percent by weight. It will be recognized that the effective concentration is also a function of the contact time between the food product and the sanitizing liquid.

The sanitizing solution preferably contains ozone, at a concentration up to 15 ppm and preferably from 0.1 to 5 ppm. Ozone solutions can be formed by means conventional in the art, wherein station 5 designates a conventional ozone generator that generates a gaseous stream of ozone which is sparged into a stream or tank of water so as to form a solution having the desired concentration of ozone in the water.

The sanitizing solution can optionally but preferably contain a surfactant which in the amount used is physiologically nontoxic, in an amount effective to increase the effectiveness of the sanitizing liquid in dislodging microbes from the surface of the food product when used as described herein. Examples of suitable surfactants for this purpose include polyethylene glycol (PEG) having a molecular weight of 100 to 100,000. The amount of surfactant present in the sanitizing liquid can be in the range of 0.01 wt. % to 0.5 wt. %.

One significant and unexpected advantage of the combination of ozone with the antimicrobial agent is that the combination in the sanitizing liquid provides rapid sanitizing, shelf life prolongation, and retention of the fresh appearance of the food product, with the use of less of the ozone and less of the agent than would be expected if either were used without the other.

The sanitizing liquid is preferably applied to all external surfaces of the food product, at a velocity which is sufficient to dislodge microbes from the surface of the food product. Suitable velocities are at least 20 feet per second up to about 200-250 feet per second at the food product surface. Too slow a velocity fails to dislodge microbes sufficiently, and too high a velocity damages the surface of the food product. When the sanitizing agent comprises ozone or another compound that easily leaves solution, a low velocity is preferred so as to avoid losing too much of the compound from the solution before it has had a full opportunity to sanitize the surface of the product. Thus, a suitable velocity for applying a solution of ozone is on the order of 25 feet per second.

The sanitizing liquid can be applied in a plurality of sprays to the surface of the food product, at a velocity sufficient so that at the points at which the liquid impinges on the surface of the food product, microbes are dislodged from the surface of the food product. By “sprays” is meant any flow of the liquid, whether applied through a wide angle or in a narrow stream or jet, by which the liquid impinges on the food product surface at sufficient velocity to achieve the objects described herein.

The sanitizing liquid is applied while the food product is moving laterally with respect to the sprays. By “laterally” is meant that the food product is moving at an angle to the axis of the spray, rather than moving only directly toward or away from the spray.

Specific apparatus useful in this regard depends somewhat upon the geometry of the food product. Relatively round products can be moved by a roller-bar type of conveyor or a relatively conventional conveyor system under, over, or through a multi-nozzle spray manifold. Smaller products can be treated using a multi-step flighted conveyor, passing through several spray manifolds. Cut produce and similarly shaped food products cut from animal carcasses can be treated in a device presenting a rotating drum, which presents the additional feature that continuous feeding of the product can be effected through the drum, with the sanitizing liquid being applied via an internal spray manifold. Such a system can also be operated on a batch basis. It will be noted that movement of the food product relative to the spray can be effected by moving the food product while the spray remains stationary, moving the spray while the food product remains stationary, or moving both.

In a preferred embodiment, the food product is carried on a conveyor (such as an endless belt) which has slots or openings to permit air and liquid to pass through the conveyor, while the conveyor passes between sprays 6 and 7 located above and below the conveyor. Preferably, in such an arrangement there are at least two sprays above the conveyor and at least two sprays below the conveyor.

Following passage of the food product in contact with the sprays of the sanitizing liquid, additional contact time of the sanitizing liquid with the food product on the order of at least 5 seconds, preferably at least 30 seconds, should be provided. This can be provided by continuing to have the food product travel along a conveyor, by holding the food product at a holding station, or otherwise as desired by the operator. This time permits effective sanitizing in that sanitizing liquid is able to achieve maximal wetting and penetration of the surfaces of the food product while the sanitizing agent is still capable of effecting sanitizing (that is, before the agent has been consumed). That is, the solution acts at the exterior surface of the food product and is also able to penetrate into any interstices of the food product. This interstitial penetration is another unexpected advantage of the present invention in that operation with the sanitizing liquid according to this invention permits the sanitizing liquid to penetrate farther into interstices of the food product, while still able to sanitize, than has been attainable with other liquid sanitizing compositions.

The sanitizing liquid is then removed from the food product at station 10.

A nozzle or two or more nozzles at 11 are positioned over conveyor 1 so as to direct a flow of a gaseous stream toward articles carried on conveyor 1. Preferably, nozzle 11 spans the width of the conveyor. The gaseous stream can comprise species of the sanitizing agent, especially where the sanitizing agent includes ozone. When the gaseous stream comprises gaseous ozone, typically in amounts that can range from 0.01 ppm to 1.0 ppm. The remainder of the gaseous stream can be air, or other gaseous carriers such as argon or nitrogen. Air is preferred because it is plentiful and free. The nozzle(s) should be close enough to the product's upper surface that the gaseous stream contacts the surface of the product.

Vacuum dewatering plenum 12 is also provided, and is preferably positioned below the surface of conveyor 1. Thus, it can be seen that a greatly preferred construction of conveyor 1 is any in which air and liquid can pass through conveyor 1. The mouth of vacuum dewatering plenum 12 is preferably configured to collect liquid that is drawn off of product passing by on conveyor 1. Thus, the mouth of vacuum dewatering plenum 12 preferably extends the entire width of conveyor 1, from side to side thereof. Vacuum dewatering plenum 12 also draws in the gaseous stream that emanated from nozzle 11, after that gaseous stream has passed around the surfaces of articles on conveyor 1.

The off-stream which vacuum dewatering plenum 12 collects thus comprises liquid such as water, containing any residual sanitizing agent such as ozone mixed or dissolved therein, and further comprises the gaseous stream which can also contain residual sanitizing agent including gaseous ozone. This off-stream is conveyed via line 15 to dewatering separator station 13, where liquid water is removed from the off-stream using any technique of the many that are well known in engineering practice (such as velocity reduction and/or demisters). As much liquid water as possible should be removed from the off-stream in dewatering separator station 13. Preferably, most of the liquid water and small droplets are removed. The liquid water exits dewatering separator station 13 as stream 14 for disposal or reuse by reinjection. This stream may contain residual amounts of sanitizing agent such as ozone and has thereby been sanitized for re-injection back into the washer and food contact.

The dewatered off-stream can comprise ozone or other sanitizing compounds, the other gaseous components that entered the dewatering separator station 13 such as air, and may also comprise water vapor. Preferably, the content of ozone and other sanitizing compounds in the dewatered off-stream that exits dewatering separator station 13 via line 18, is higher than the content thereof in the off-stream 15 that entered dewatering station 13. That is, for instance, it is preferred that ozone and other sanitizing compounds transfers from the liquid phase of off-stream 15 into the gaseous stream that exits dewatering station 13.

This dewatered gaseous off-stream travels via line 18 around to nozzle 11, to be applied to articles on conveyor 1.

Pressure to generate the desired vacuum at the mouth of vacuum plenum 12, and any desired pressure to impinge the gaseous stream from nozzle 11 toward articles on conveyor 1, is provided in known manner by any suitable blower or fan 24 designed for food applications. As is well known, such apparatus should be constructed of material such as stainless steel, and should be capable of being opened up to reveal its interior for periodic cleaning. The fan or blower can be a separate unit, such as is depicted in phantom lines as 24 in FIG. 2. In the embodiment in FIG. 2, the gas flow is provided by a blower which is incorporated integrally into dewatering station 13. For example, a unit which integrally provides dewatering and suitable gas flow via a blower is commercially available, known as a Reyco Systems, Inc. (Meridian, Id. 83680) model “Water Vac®” System comprising a stainless steel fan and water/gas separator.

It is highly desirable to maintain control of the temperature of the streams cycling through lines 15 and 18. The temperature of the stream that is directed at the product from nozzle 11 should not exceed a predetermined maximum, so as to protect the product from being overheated. A preferred maximum temperature is 40° F., even 35° F.

One technique for controlling the maximum temperature of the stream directed from nozzle 11 is periodically or continuously introducing cooling water via line 19 through valve 21 into line 15. Other techniques will be apparent to the knowledgeable practitioner, such as passing stream 15 or stream 18 through a heat exchanger that chills the stream.

It is preferred to provide a side stream 16 that carries a portion (typically 10-30 vol. %) of the stream in line 18 out of the housing. The side stream can be discharged to the atmosphere outside the housing, but preferably the side stream is discharged into a means 17 that removes sanitizing agent from the side stream. Removal can be effected by chemical reaction, by absorption onto or into an absorbing medium, or by other techniques which are known in this field. For instance, if the side stream contains ozone, the side stream is preferably discharged into an ozone destroying station 17 in which ozone in side stream 16 is decomposed to oxygen by any known conventional means such as catalytic conversion.

Providing such a side stream 16, and providing for suitable control of the flow rate that is permitted through valve 20 into side stream 16, enables maintaining a pressure within the housing that is less than the pressure of the atmosphere outside the housing. In this way, a slight pressure differential is maintained which causes ambient air to enter the housing through the entrance 22, the exit 23, and any other openings. This pressure differential prevents gaseous disinfecting agent, such as ozone, from emerging through those openings to the areas around the outside of the housing where the agent might contact workers located there and injure them.

The method and apparatus of the present invention provide numerous advantages. One advantage, described above, is the prevention of gaseous sanitizing agent from escaping to the areas around the exterior of the housing. Another advantage is that the ozone or other sanitizing agent that is present in the gaseous stream which is applied from nozzle 11 creates a protective atmosphere that inhibits introduction or reintroduction of undesired bacteria and other material onto the food surfaces. Indeed, the gaseous stream itself which participates in removing liquid from the food product at station 10 is already rendered essentially aseptic, because of the presence of the gaseous ozone in this gaseous stream.

Another advantage is that removing water from the product using the gaseous stream directed from nozzle 11, which has been dewatered but is humid, causes less undesired dehydration of the product itself than is the case when water is removed by use of a high-velocity stream of thoroughly dried air. The system also consumes less energy by conserving cooled ambient air within the housing.

1. A method for sanitizing an article comprising
(a) providing a housing having an entrance through which an article to be sanitized can enter the housing, an exit from which the article can exit the housing, and means for transporting the article within said housing from said entrance to said exit,
(b) applying an aqueous sanitizing solution comprising a sanitizing agent to the article within said housing, and then
(c) directing a gaseous stream into contact with a surface of the article within said housing and drawing said gaseous stream past a surface of said article in a manner which draws water off of the article and into the gaseous stream drawn past said article, to form an off-stream comprising said gaseous stream and said water drawn off of said article,
(d) removing liquid water from said off-stream, thereby producing a dewatered gaseous stream,
(e) recycling said dewatered gaseous stream to step (c) to comprise the gaseous stream directed toward a surface of the article in step (c),
(f) cooling one or both of said dewatered gaseous stream and said water drawn off of said article as necessary to prevent the temperature of said gaseous stream from exceeding a predetermined maximum temperature, and
(g) withdrawing a gaseous side stream from said dewatered gaseous stream within said housing as necessary to enable the atmospheric pressure within said housing to remain less than the atmospheric pressure outside said housing, and discharging said side stream outside said housing.

2. A method according to claim 1 wherein the article is a food product.

3. A method according to claim 1 wherein said means for transporting the article comprises a conveyor that has openings in it through which air and liquid can pass.

4. A method according to claim 3 wherein in step (b) said aqueous sanitizing solution is applied from above the conveyor and in step (c) said gaseous stream is drawn off from below the conveyor.

5. A method according to claim 4 wherein in step (c) said gaseous stream is directed into contact with a surface of the article from above the conveyor.

6. A method according to claim 1 wherein said predetermined maximum temperature is 40F.

7. A method according to claim 1 wherein said side stream withdrawn in step (g) is discharged into a means for removing disinfecting agent from said stream.

8. A method for sanitizing an article comprising
(a) providing a housing having an entrance through which an article to be sanitized can enter the housing, an exit from which the article can exit the housing, and means for transporting the article within said housing from said entrance to said exit,
(b) applying an aqueous sanitizing solution comprising ozone to the article within said housing, and then
(c) directing a gaseous stream comprising ozone into contact with a surface of the article within said housing and drawing said gaseous stream past a surface of said article in a manner which draws water off of the article and into the gaseous stream drawn past said article, to form an off-stream comprising said gaseous stream comprising ozone and said water drawn off of said article,
(d) removing liquid water from said off-stream, thereby producing a dewatered gaseous stream that comprises ozone,
(e) recycling said dewatered gaseous stream to step (c) to comprise the gaseous stream directed toward a surface of the article in step (c),
(f) cooling one or both of said dewatered gaseous stream and said water drawn off of said article as necessary to prevent the temperature of said gaseous stream from exceeding a predetermined maximum temperature, and
(g) withdrawing a gaseous side stream comprising ozone from said dewatered gaseous stream within said housing as necessary to enable the atmospheric pressure within said housing to remain less than the atmospheric pressure outside said housing, and discharging said side stream outside said housing.

9. A method according to claim 8 wherein the article is a food product.

10. A method according to claim 8 wherein said means for transporting the article comprises a conveyor that has openings in it through which air and liquid can pass.

11. A method according to claim 10 wherein in step (b) said aqueous sanitizing solution is applied from above the conveyor and in step (c) said gaseous stream is drawn off from below the conveyor.

12. A method according to claim 11 wherein in step (c) said gaseous stream is directed into contact with a surface of the article from above the conveyor.

13. A method according to claim 8 wherein said predetermined maximum temperature is 40F.

14. A method according to claim 8 wherein said side stream withdrawn in step (g) is discharged into a means for removing disinfecting agent from said stream.

15. Apparatus useful for sanitizing an article comprising
(a) a housing having an entrance through which an article to be sanitized can enter the housing, an exit from which the article can exit the housing, and means for transporting the article within said housing from said entrance to said exit,
(b) means for applying an aqueous sanitizing solution comprising a sanitizing agent to the article within said housing,
(c) means within said housing for directing a gaseous stream into contact with a surface of the article to which said aqueous sanitizing solution has been applied,
(d) means for drawing said gaseous stream past a surface of said article in a manner which draws water off of the article and into the gaseous stream drawn past said article, to form an off-stream comprising said gaseous stream and said water drawn off of said article,
(e) means for removing liquid water from said off-stream, thereby producing a dewatered gaseous stream,
(f) means for recycling said dewatered gaseous stream to step (c) to comprise the gaseous stream directed toward a surface of the article in step (c),
(g) (i) means for cooling said dewatered gaseous stream, or (ii) means for cooling and said water drawn off of said article, or (iii) both means (i) and means (ii), wherein said means (i), (ii) or (iii) is capable of cooling sufficiently to prevent the temperature of said gaseous stream from exceeding a predetermined maximum temperature, and
(h) means for withdrawing a gaseous side stream from said dewatered gaseous stream within said housing as necessary to enable the atmospheric pressure within said housing to remain less than the atmospheric pressure outside said housing, and for discharging said side stream outside said housing.

16. An apparatus according to claim 15 wherein said means for transporting the article comprises a conveyor that has openings in it through which air and liquid can pass.

17. An apparatus according to claim 16 wherein means (b) applies said aqueous sanitizing solution from above the conveyor and means (c) draws off said gaseous stream from below the conveyor.

18. An apparatus according to claim 17 wherein means step (c) directs said gaseous stream into contact with a surface of the article from above the conveyor.

19. A method according to claim 15 wherein said predetermined maximum temperature is 40F.

20. A method according to claim 15 wherein said side stream withdrawn in step (g) is discharged into a means for removing disinfecting agent from said stream.
